(19) United States
(12) Patent Application Publication
(10) Pub. No.: US 2025/0205247 A1
(43) Pub. Date: Jun. 26, 2025

(54) USE OF BAZEDOXIFENE FOR INCREASING MUSCLE SURVIVAL

(71) Applicants: CENTRE D'ETUDE DES CELLULES SOUCHES, Corbeil Essonnes (FR); GENETHON, Evry-Courcouronnes (FR); UNIVERSITE D’EVRY-VAL-D’ESSONNE, Evry-Courcouronnes (FR); INSERM (INSTITUT NATIONAL DE LA SANTE ET DE LA RECHERCHE MEDICALE), Paris (FR)

(72) Inventors: Xavier NISSAN, Charenton-Le-Pont (FR); Céline BRUGE, Grisy Suisnes (FR); Emilie PELLIER, Epinay Sur Orge (FR); Isabelle RICHARD, Corbeil Essonnes (FR)

(21) Appl. No.: 18/846,458

(22) PCT Filed: Mar. 15, 2022
(86) PCT No.: PCT/EP2023/056619

§ 371 Date: Sep. 12, 2024

(30) Foreign Application Priority Data
Mar. 15, 2022 (FR) FR2202266

(51) Int. Cl. A61K 31/55; A61P 21/00

(52) U.S. Cl. CPC A61K 31/55; A61P 21/00

(57) ABSTRACT

The invention concerns the use of bazedoxifene as a representative of third generation SERMs, namely compounds comprising a 2-phenylindole group, in order to increase muscle survival, in particular in the treatment of dysferlinopathies.

### FIELD OF THE INVENTION

The aim of the present invention is to improve muscle function in human beings or animals by preserving the integrity and survival of muscle cells, even under unfavorable conditions.

More precisely, it recommends the use of bazedoxifene, a selective estrogen receptor modulator (or SERM), to ensure the protection of muscle function, in particular in pathological conditions such as muscular dystrophies.

### PRIOR ART

Neuromuscular diseases encompass various pathologies that are generally associated with a temporary or permanent loss of muscle strength. This loss of strength is usually accompanied by muscle wasting, also known as amyotrophy.

Among these muscle diseases, myopathies constitute an important group corresponding to attacks on the muscle fiber per se. Of these. progressive muscular dystrophies are characterized by a reduction in muscle strength, generally with atrophy of the muscles. as well as by anomalies in the muscle biopsy, revealing a tissue modification. Duchenne muscular dystrophy (or DMD), Becker muscular dystrophy (or BMD) as well as limb-girdle muscular dystrophies in particular belong to this group.

For some of these pathologies, the associated genetic anomalies have been able to be identified. Thus, Duchenne or Becker muscular dystrophies are linked to alterations in the gene coding for dystrophin, limb-girdle muscular dystrophy type 2A (LGMD 2A or R1 or calpainopathy) to alterations in the gene for calpain 3, dysferlinopathy (LGMD 2B or R2) to alterations in the gene for dysferlin, sarcoglycanopathies or limb-girdle muscular dystrophies type LGMD 2C (or R5), LGMD 2D (or R3), LGMD 2E (or R4), LGMD 2F (or R6), to deficiencies in the genes for γ-, α-, β- and δ-sarcoglycans, respectively (McNally E M, Pytel P, Annu Rev Pathol. 2007: Vol 2:87-109).

In respect of dysferlinopathy, it has been shown that the mutation L1341P in the gene for dysferlin results in a poor conformation of the resulting protein, associated with its aggregation in the endoplasmic reticulum. The absence of dysferlin in the sarcolemma then causes membrane fragility, which in the end causes the death of muscle cells and therefore participates in muscular dystrophy.

Various gene therapy strategies are being developed for these pathologies, but they remain difficult to implement.

However, and more generally in all cases of muscle weakness, there is an evident need for the development of technical solutions that can preserve muscle function, in particular with the aid of chemical compounds, which are easier to master than gene therapy.

Thus, the present invention resides in the demonstration of this property of bazedoxifene by the inventors.

The document WO 2009/021750 describes the use of the compound (2-phenyl-1H-indol-3-yl) methanol, which is not a SERM, linked to the treatment of Duchenne or Becker muscular dystrophies and of cachexia.

The document WO 2015/108988 describes the use of a combination of a SERM (advantageously lasoxifen, which does not comprise a 2-phenylindole group) and a 5α-reductase inhibitor in order to treat any pathology linked to a modification in sex hormone levels.

The document XIA HUI et al. (PLOS ONE, 2017, Vol. 12(7)) describes the use of bazedoxifene for the specific treatment of rhabdomyosarcoma.

The document WANG JING et al. (FRONTIERS IN PHARMACOLOGY, 2021, Vol. 11, pages 1-13) describes the use of bazedoxifene for the treatment of a specific inflammatory cardiomyopathy, namely auto-immune myocarditis.

The document DORCHIES et al. (JOURNAL OF NEUROMUSCULAR DISEASES, 2014, Vol. 1(1)) reports the beneficial effect of tamoxifen and other first or second generation SERMs on a mouse model of Duchenne muscular dystrophy.

The document WU BO et al. (THE AMERICAN JOURNAL OF PATHOLOGY, 2018, Vol. 188(4), pages 1069-80) reports the beneficial effect of the oral administration of tamoxifen (1st generation SERM) and raloxifene (2^{nd} generation SERM) in a mouse model of dystroglycanopathy.

### DETAILED DESCRIPTION OF THE INVENTION

The inventors have shown that bazedoxifene, a pharmacological compound known principally for the prevention and treatment of post-menopausal osteoporosis because of its action as a selective estrogen receptor modulator (or SERM), is a promising candidate for the treatment of dysferlinopathies. The present application shows that it is effective in a wide range of conditions associated with fragility of the muscle cells, in particular linked to the integrity of their membranes.

### Definitions

The definitions below provide the general meaning used in the context of the invention and should be applied, unless another definition is explicitly indicated.

In the context of the invention, the articles “a” and “an” are used to designate one or more (i.e. at least one) of the grammatical objects of the article. By way of example, “an element” designates at least one element, i.e. one or more elements.

The terms “about”, “nearly”, “of the order of” or “approximately” used in the present document to designate a measurable value such as a quantity, a duration and others, should be understood to encompass variations of ±20% or ±10%, preferably ±5%, more preferably ±1%, and yet more advantageously ±0.1% with respect to the specified value.

Intervals/ranges: throughout this disclosure, various aspects of the invention may be presented in the form of an interval of values (forming a range). It should be understood that the description of the values in the form of an interval is only provided for convenience and brevity and should not be interpreted as a limitation to the scope of the invention. As a consequence, the description of a range should be considered to have specifically disclosed all possible sub-ranges as well as the individual numerical values within that range. As an example. the description of a range as “from 1 to 6” should be considered to specifically disclose sub-ranges such as from 1 to 3, from 1 to 4, from 1 to 5, from 2 to 4, from 2 to 6, from 3 to 6, etc. as well as the individual numbers within that range, for example 1, 2, 2.7, 3, 4, 5, 5.3 and 6. This applies irrespectively of the extent of the range.

“Isolated” signifies extracted or withdrawn from its environment or natural state. As an example, a nucleic acid or an isolated peptide is a nucleic acid or a peptide which has been extracted from the natural environment in which it is normally found, irrespectively of whether it is a plant or a living animal, for example. A nucleic acid or a peptide, for example, which is naturally present in a living animal is not a nucleic acid or an isolated peptide within the meaning of the invention, although the same nucleic acid or peptide partially or completely separated from the other components present in its natural environment is itself “isolated” within the meaning of the invention. A nucleic acid or an isolated protein may exist in a substantially purified form, or it may exist in a non-native environment such as a host cell, for example.

The term “abnormal”, when used in the context of organisms, tissues, cells or components thereof, designates organisms, tissues, cells or components thereof which differ in at least one observable or detectable characteristic (for example age, treatment, hour of the day, etc) from organisms, tissues, cells or components thereof which have the respective “normal” (expected) characteristic. Characteristics that are normal or expected for one type of cell or tissue may be abnormal for another type of cell or tissue.

The terms “patient”, “subject”, “individual” and others are used interchangeably in the present document and designate any animal or cell thereof. in vitro or in situ, which could be exposed to the methods described in the present document. In certain non-limiting embodiments, the patient, the subject or the individual is an animal, preferably a mammal. more advantageously a human being, of either the female or the male sex. It may also be a mouse, a rat, a pig, a dog or a non-human primate (NHP) such as a macaque monkey.

Within the meaning of the invention, a “disease” or “pathology” is a state of health of an animal in which their homeostasis is negatively affected and which continues to deteriorate if the disease is not treated. In contrast, withing the meaning of the invention, a “problem” or “dysfunction” is a state of health in which the animal is capable of maintaining their homeostasis, but in which the state of health of the animal is less favorable than it would be in the absence of a problem. In the absence of treatment, a problem does not necessarily result in a deterioration in the state of health of the animal over time.

A disease or a problem is “alleviated” (“reduced”) or “improved” if the severity of a symptom of the disease or the problem, the frequency at which that symptom is experienced by the subject. or both. is reduced. This also includes the disappearance of the progress of the disease. i.e. the progress of the disease or the problem stops. A disease or a problem is “healed” (“recovered from”) if the severity of a symptom of the disease or the problem, the frequency at which such a symptom is experienced by the patient, or both, is eliminated.

In the context of the invention, a “therapeutic” treatment is a treatment administered to a subject who presents symptoms (signs) of a pathology. with the aim of reducing or removing those symptoms. In the context of the invention, the “treatment of a disease or a dysfunction” means the reduction in the frequency or the severity of at least one sign or symptom of a disease or of a dysfunction experienced by the subject. A treatment is said to be prophylactic when it is administered in order to prevent the development, propagation or aggravation of a disease, in particular if the subject does not present or does not yet present the symptoms of the disease and/or for whom the disease has not yet been diagnosed.

As used here, “to treat a disease or a dysfunction” means reducing the frequency or the severity of at least one sign or symptom of a disease or a dysfunction experienced by a subject. Disease and dysfunction are used interchangeably in the context of the treatment in accordance with the invention.

Within the meaning of the invention, an “efficacious quantity” or an “effective quantity” of a compound is the quantity of compound which is sufficient to provide a beneficial effect to the subject to whom the compound is administered. The expression “therapeutically effective quantity” refers to a quantity that is sufficient or effective in preventing or treating (in other words retarding or inhibiting development, inhibiting the progression, hindering, reducing, diminishing or reversing) a disease or a dysfunction, including relieving the symptoms of that disease or that dysfunction.

Thus, the present invention concerns the use of a specific selective estrogen receptor modulator (SERM), advantageously of third generation, i.e. comprising a 2-phenylindole group, yet more advantageously bazedoxifene or its derivatives, in order to combat fragility or even the death of muscle cells, and therefore to increase muscle survival.

Selective estrogen receptor modulators (or SERM) are non-steroidal molecules that are capable of binding to estrogen receptors. They have both agonist and antagonist properties as regards estrogens. Such molecules can be identified using well-known assays such as the “estrogen response element (ERE) transactivation assay”, or as described by Nelson (2016, Methods Mol Biol: 431-443).

In practice, various compounds having such an activity are available:

What are known as first generation SERMs are derivatives of triphenylethylene.

Such compounds may, for example, be selected from:
Tamoxifen, with formula:

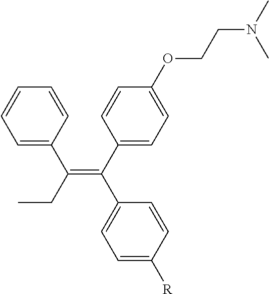

tamoxifen citrate
clomifene or enclomifene or clomifene citrate (CLOMID)
toremifene or toremifene citrate
afimoxifene (4-hydroxytamoxifen)
levormeloxifene or ormeloxifene
droloxifene
idoxifene
fispemifene
ospemifene ((deaminohydroxy)toremifene), with formula:

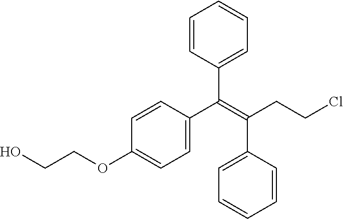 [SMILES: OCCOc1ccc(/C(=C(/CCCl)c2ccccc2)c2ccccc2)cc1]

endoxifene, with formula:

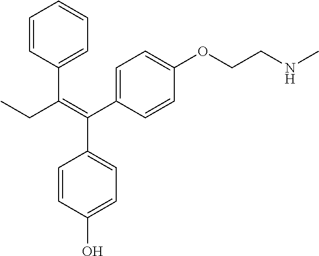 [SMILES: CC/C(=C(\c1ccc(O)cc1)c1ccc(OCCNC)cc1)c1ccccc1]

What are known as second generation SERMs are derivatives of benzothiophene, for example:
raloxifene, with formula:

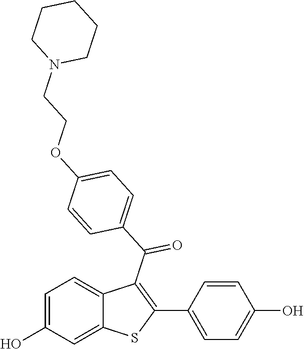 [SMILES: O=C(c1ccc(OCCN2CCCCC2)cc1)c1c(-c2ccc(O)cc2)sc2cc(O)ccc12]

arzoxifene

What are known as third generation SERMs are derivatives of 2-phenylindole with formula:

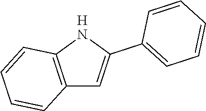

Such compounds may, for example, be selected from:
bazedoxifene (CAS number: 198481-32-2), with formula:

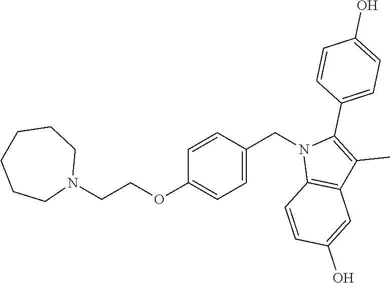 [SMILES: Cc1c(-c2ccc(O)cc2)n(Cc2ccc(OCCN3CCCCCC3)cc2)c2ccc(O)cc12]

bazedoxifene acetate, with formula:

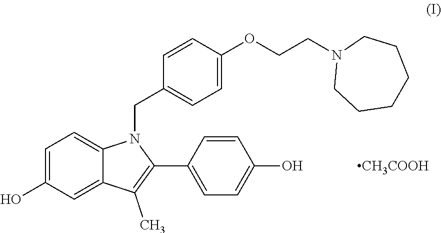

zindoxifene, with formula:

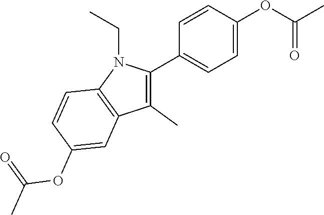 [SMILES: CCn1c(-c2ccc(OC(C)=O)cc2)c(C)c2cc(OC(C)=O)ccc21]

pipendoxifene, with formula:

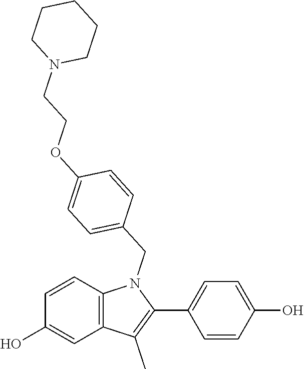 [SMILES: Cc1c(-c2ccc(O)cc2)n(Cc2ccc(OCCN3CCCCC3)cc2)c2ccc(O)cc12]

Other SERMs that are of potential interest are:
Nafoxidine, with formula:

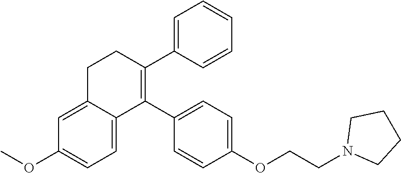 [SMILES: COc1ccc2c(c1)CCC(c1ccccc1)=C2c1ccc(OCCN2CCCC2)cc1]

Lasofoxifene, with formula:

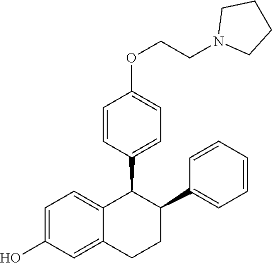 [SMILES: Oc1ccc2c(c1)CC[C@H](c1ccccc1)[C@@H]2c1ccc(OCCN2CCCC2)cc1]

Preferably, the compound employed in the context of the present invention is a SERM and/or comprises a 2-phenylindole group with formula:

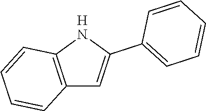

Advantageously, it is selected from the group constituted by: bazedoxifene, bazedoxifene acetate, zindoxifene, pipendoxifene, their derivatives and/or their combinations.

Alternatively, it may be ospemifene, endoxifene or raloxifene.

In accordance with a particular embodiment, it is not tamoxifen, nor is it fispemifene, nor is it raloxifene.

Yet more advantageously, it is bazedoxifene or bazedoxifene acetate.

Bazedoxifene has been approved by the FDA and ANSM for the prevention of post-menopausal osteoporosis. This drug, marketed under the trademark Conbriza®, is in the form of tablets comprising 20 mg of bazedoxifene acetate (with a tablet core containing: Lactose monohydrate (142.8 mg)+Microcrystalline cellulose+Pregelatinized (com) starch+Sodium starch glycolate+Sodium lauryl sulfate+Anhydrous colloidal silica+Magnesium stearate+Ascorbic acid, and a coating containing: Hypromellose+Titanium dioxide (E171)+Macrogol 400), 1 tablet to be taken orally and daily.

The present invention also pertains to derivatives of these compounds, in particular bazedoxifene, with the same biological activity, in particular that of a SERM or that reported in the examples, for example on the survival of muscle cells under osmotic shock conditions.

The term “derivatives” encompasses derivatives and metabolites, as well as pharmaceutically acceptable salts. A derivative is a compound which originates from another compound (the precursor, typically with a similar chemical structure) after transformation of the latter. The derivative may differ by one or more atoms or functional groups. A metabolite is a stable intermediate compound or a compound resulting from the biochemical transformation of an initial molecule by metabolism.

The term “pharmaceutically acceptable salts” means addition salts of a compound, which may be obtained by reaction of this compound with a mineral or organic acid in accordance with a method that is known per se. Acids that may be cited which may be used for this purpose include hydrochloric (HCl), hydrobromic, sulfuric, phosphoric, 4-toluene sulfonic, methane sulfonic, cyclohexyl sulfamic, oxalic, succinic, formic, fumaric, maleic, citric, aspartic, cinnamic, lactic, glutamic acids. N-acetyl-aspartic, N-acetyl-glutamic, ascorbic, malic, benzoic, nicotinic and acetic acids as well. By way of example, bazedoxifene acetate is routinely used in the pharmaceutical field.

Esters on the hydroxy function that may be cited include esters of carboxylic acids containing 1 to 6 carbon atoms.

Said compounds, in particular bazedoxifene, may be modified in order to increase their stability, their bioavailability and/or their capacity to reach target tissues, in particular muscle tissues and cells.

In a manner that is known to the person skilled in the art, said compounds, in particular bazedoxifene, may be present in the composition in the naked (free) form or contained in delivery systems which increase stability, targeting and/or bioavailability, such as liposomes, or incorporated into supports such as hydrogels, cyclodextrins, biodegradable nanocapsules, bioadhesive microspheres, vectors or in combination with a cationic peptide.

The present invention also concerns pharmaceutical compositions containing, as the active principle, at least one compound as defined above, as well as the use of this compound or of this composition as a drug or medicinal product. In accordance with a particular embodiment, the compound in accordance with the invention is the only therapeutic agent present in the pharmaceutical composition.

Thus, the present invention pertains to pharmaceutical compositions comprising a compound in accordance with the invention. Advantageously, these compositions comprise a therapeutically effective quantity of said compound, and a pharmaceutically acceptable support. In a particular embodiment, the term “pharmaceutically acceptable” means approved by a regulating organization of a federal government or state or listed in the American or European pharmacopoeia or in another generally recognized pharmacopoeia for use in animals and human beings. The term “support” designates a diluent, an adjuvant, an excipient or a vehicle with which the therapeutic product is administered. These pharmaceutical supports may be sterile liquids, such as water and oils, including those of oil, animal, vegetable or synthetic origin, such as groundnut oil, soy bean oil, mineral oil, sesame seed oil and others. Saline solutions and aqueous solutions of dextrose and glycerol may also be employed as liquid supports, in particular for injectable solutions. Appropriate pharmaceutical excipients include starch, glucose, lactose, saccharose, sodium stearate, glycerol monostearate, talc, sodium chloride, powdered skimmed milk, glycerol, propylene glycol, water, ethanol and others.

If necessary, the composition may also contain minor quantities of wetting agents or emulsifiers, or pH buffering agents. These compositions may take the form of solutions, suspensions, emulsions, slow-release formulations and others. Examples of appropriate pharmaceutical supports are described in “Remington's Pharmaceutical Sciences” by E. W. Martin. These compositions contain a therapeutically effective quantity of therapeutic agent, preferably in the purified form, as well as an appropriate quantity of support in a manner such as to provide the form that allows for suitable administration to the subject.

In a preferred embodiment, the composition is formulated in accordance with routine protocols, such as a pharmaceutical composition which is suitable, for example, for oral administration to human beings. Typically, the compositions for oral administration are in the form of tablets, optionally scored tablets or effervescent tablets, further containing excipients which are adapted to the solid dosage form and to administration to human beings. By way of example, the commercial forms of bazedoxifene which are available are tablets which also contain lactose monohydrate, microcrystalline cellulose, pregelatinized (corn) starch, sodium starch glycolate, sodium lauryl sulfate, anhydrous colloidal silica, magnesium stearate and ascorbic acid. These tablets may be crushed and mixed with liquids.

Alternatively, the composition may be in a liquid form, advantageously an aqueous composition. Any other appropriate solvent may be used.

The quantity of therapeutic agent of the invention, i.e. a compound as described above, which will be effective in the treatment of a disease may be determined using standard clinical techniques. Furthermore, in vivo and/or in vitro assays, such as those described in the examples below, may optionally be used in order to aid in predicting the optimal dosage ranges. The precise dose to be used in the formulation will also depend on the administration route, the weight and the severity of the disease, and must be decided using the judgement of the clinician and the conditions for each patient.

In accordance with a particular embodiment, the composition of the invention is in a solid form, advantageously a tablet, comprising 20 mg of active compound, in particular bazedoxifene or bazedoxifene acetate, or even less. Preferably, the composition comprises a quantity equal to or less than 20 mg, 15 mg, 10 mg, or even equal to or less than 5 mg.

In accordance with another embodiment, the composition of the invention is in a liquid form and advantageously comprises less than 30 μM or 10 μM of active compound, in particular bazedoxifene or bazedoxifene acetate, more advantageously between 30 nM and 10 μM, yet more advantageously between 100 nM and 5 μM, or even between 300 nM and 2 μM.

An appropriate administration route should allow a therapeutically effective quantity of therapeutic product to be delivered to the target cells, depending on the disease.

The administration routes that are available are as follows: topical (local), enteral (systemic action, but delivered via the gastro-intestinal (GI) tract, or parenteral (systemic action, but delivered by routes other than the GI tract). In the specific case of muscle diseases. the preferred administration route for the compositions disclosed here is generally enteral. which includes oral administration. In accordance with other embodiments, this may be a parenteral administration, in particular intramuscular (i.e. into the muscle) or systemic (i.e. into the circulatory system). In this context, the term “injection” (or “perfusion” or “infusion”) encompasses intravascular administration, in particular intravenous (IV), and intramuscular (IM). The injections are generally carried out using syringes or catheters.

In accordance with one embodiment, the composition is administered orally. intramuscularly, intraperitoneally. subcutaneously, topically, locally or intravascularly. advantageously orally. In accordance with a preferred embodiment, the composition is intended for oral administration.

As already mentioned, a composition in accordance with the invention is preferably in a solid galenical form adapted for oral administration, advantageously in the form of one or more capsules or tablets. Thus, they may be taken with a little water before or during the main meal. In accordance with a preferred embodiment, the composition in accordance with the invention is administered daily, for example once a day. The treatment may be for several weeks, several months, several years or even lifelong.

As already indicated, the patient is advantageously a human being, in particular a newborn. a young child, a child, an adolescent or an adult. irrespective of their sex. The therapeutic tool in accordance with the invention may, however, be adapted and used to treat other animals, in particular pigs, mice, dogs or the macaque monkey.

In general, the aim of the present invention is to combat (or reduce or alleviate or preserve) muscle cell, fiber or tissue mortality and/or fragility, in other words to improve (or increase or preserve) muscle survival and/or strength and/or function. In fact, and as demonstrated in the present application, the compounds described above will improve membrane resistance, in particular to stress, for example under osmotic shock conditions or after laser irradiation.

In the context of the invention, the terms “muscle” and “muscular” advantageously pertain to the striated muscles, yet more advantageously to the skeletal muscles. In accordance with a particular embodiment, the cardiac muscle is not concerned.

Thus, and as already mentioned. the present invention concerns the treatment of diseases associated with a muscle fragility, in particular an instability of the membrane of muscle cells. In other words, the present invention can contribute to improving muscle survival, and as a consequence muscle function, in particular muscle strength.

In accordance with a particular embodiment, the skeletal muscles are mainly or even exclusively affected. Thus, the cardiac muscle may be spared the disease.

As illustrated in the examples, muscle fragility, in particular of the membranes of muscle cells, can be evaluated with the aid of in vitro tests under osmotic shock conditions.

Thus, and in a more precise manner. the invention also pertains to:
a composition comprising a compound as described above, in particular bazedoxifene as a representative of third generation SERMs, namely compounds comprising a 2-phenylindole group, in order to treat pathologies associated with a muscle fragility:
the use of a compound as described above, in particular bazedoxifene as a representative of third generation SERMs, namely compounds comprising a 2-phenylindole group, for the preparation of a drug intended to treat pathologies associated with a muscle fragility:
a method for treating pathologies associated with a muscle fragility, comprising the administration of a compound as described above, in particular bazedoxifene as a representative of third generation SERMs, namely compounds comprising a 2-phenylindole group:
a composition comprising a compound as described above, in particular bazedoxifene as a representative of third generation SERMs, namely compounds comprising a 2-phenylindole group, in order to increase muscle survival/function/strength:
the use of a compound as described above, in particular bazedoxifene as a representative of third generation SERMs, namely compounds comprising a 2-phenylindole group, in order to increase muscle survival/function/strength:
a method for increasing muscle survival/function/strength, comprising the administration of a compound as described above, in particular bazedoxifene, as a representative of third generation SERMs, namely compounds comprising a 2-phenylindole group.

In practice, there are a certain number of conditions in which the muscle function is weakened.

Firstly, they may be pathological conditions, in particular in the case of neuromuscular diseases.

In accordance with one embodiment, the disease concerned is of genetic origin, in particular caused by one or more mutations in one or more genes responsible for said disease.

Thus, muscular dystrophies caused by mutations in the gene for dysferlin (DYSF), such as dysferlinopathy (LGMD 2B or R2), Miyoshi type distal myopathy and distal anterior compartment myopathy, are the pathologies which are particularly concerned, but all forms of neuromuscular diseases, in particular muscular dystrophies, may be treated.

As demonstrated in the examples, dysferlinopathy (LGMD 2B or R2) linked to the L1341P, G299R or S1173X mutation may therefore be treated.

In certain embodiments, a compound as described or a pharmaceutical composition comprising it is intended to be used in order to treat muscle diseases (i.e. myopathies) or muscle lesions, in particular genetic neuromuscular dysfunctions, without liver damage or even without cardiac injury, such as, for example: muscular dystrophies, congenital muscular dystrophies, congenital myopathies, distal myopathies, other myopathies, myotonic syndromes, ion channel muscle diseases, malignant hyperthermia, metabolic myopathies and other neuromuscular dysfunctions, advantageously muscular dystrophies, congenital muscular dystrophies, congenital myopathies, distal myopathies and other myopathies.

The muscular dystrophies that may therefore be treated in particular include:
dystrophinopathies, a range of muscle diseases linked to the X chromosome, caused by pathogenic variants in the DMD gene, which codes for the protein dystrophin. The dystrophinopathies include Duchenne muscular dystrophy (DMD), Becker muscular dystrophy (BMD) and dilated cardiomyopathy associated with DMD;
limb-girdle muscular dystrophies (LGMD), which are a group of problems clinically similar to DMD, but which arise in both sexes due to an autosomal recessive or autosomal dominant transmission. Limb-girdle muscular dystrophies are caused by the mutation of genes that code for sarcoglycans and other proteins associated with the muscle cell membrane, which interact with dystrophin. The term LGMD1 designates the genetic types having a dominant heredity (autosomal dominant), while the term LGMD2 designates the types having an autosomal recessive heredity. Pathogenic variants have been reported on more than 50 loci (LGMDIA to LGMDIH: LGMD2A to LGMD2Y);
calpainopathy (LGMD2A/R1) is caused by a mutation in the CAPN3 gene, with more than 450 pathogenic variants being described.

A non-limiting list of these diseases includes: centronuclear myopathy, advantageously X-linked myotubular myopathy (XLM™) and Charcot-Marie-Tooth disease, limb-girdle muscular dystrophy, advantageously LGMD2A/R1, LGMD2B/R2, LGMD2D/R3 or LGMD2I/R9, LGMDID/DI, LGMD2L/R12, congenital muscular dystrophy type 1C (MDCIC), Walker-Warburg syndrome (WWS), muscle-eye-brain disease (MEB), Duchenne muscular dystrophy (DMD) or Becker muscular dystrophy (BMD), congenital muscular dystrophy with selenoprotein N deficit, selenoprotein N-deficient congenital muscular dystrophy, primary merosin-deficient congenital muscular dystrophy, Ullrich congenital muscular dystrophy, central core congenital myopathy, multi-minicore congenital myopathy, autosomal centronuclear myopathy, fiber-type disproportion myopathy, nemaline myopathy, congenital myasthenic syndromes, Miyoshi distal myopathy, dysferlinopathies, dystroglycanopathies and sarcoglycanopathies. In some embodiments, the pharmaceutical composition of the invention is intended to be used in order to treat Duchenne muscular dystrophy (DMD) or Becker muscular dystrophy (BMD), or congenital limb-girdle muscular dystrophy, advantageously LGMD2A/R1, LGMD2B/R2, LGMD2D/R3, LGMD2I/R9, LGMDID/DI or LGMD2L/R12.

Furthermore, cachexia or marasmus is also a pathological state with which the present invention is concerned. This state is characterized by extreme thinness, in particular at the level of the skeletal muscles, caused by a long-term disease or insufficient protein or calorie intake.

This pathological state is in particular observed in the case of chronic diseases such as cancer or AIDS or in persons suffering either from cardiac insufficiency—atrophy of the skeletal muscles is observed in 68% of patients—, or urinary incontinence.

Without being considered to be pathologies in the true sense, certain situations are associated with a loss or deterioration in muscle function or strength: ageing. prolonged immobilization, etc. Thus, here too, there is an interest in improving muscle survival and therefore in increasing muscle function or strength.

Thus, the present invention resides in the demonstration of protective properties for bazedoxifene, as a representative of SERMs, in particular third generation SERMs, namely compounds comprising a 2-phenylindole group, in particular in respect of muscle cell membranes.

In accordance with a particular embodiment, the pathology treated in the context of the present invention is not rhabdomyosarcoma. In accordance with another particular embodiment, the pathology treated in the context of the present invention is not a cardiomyopathy, in particular auto-immune myocarditis.

As already mentioned, a composition comprising a compound in accordance with the invention, intended for the treatment of pathologies associated with muscle fragility or intended to improve muscle survival, may furthermore contain any acceptable, in particular pharmaceutical, compound or excipient.

This treatment may also be associated with other treatments, intended to treat the same pathology or the same state.

In order to promote grafting of precursor cells or stem cells, it may be advantageous to associate the administration of bazedoxifene as a representative of third generation SERMs, namely compounds comprising a 2-phenylindole group, with cell grafting (myoblasts, stem cells, etc.). This administration may be carried out simultaneously or at different times.

It may also be advantageous to combine a gene therapy intended for the treatment of a neuromuscular disease with the administration of a compound as described above, in particular bazedoxifene as a representative of third generation SERMs, namely compounds comprising a 2-phenylindole group. Thus, and in accordance with a particularly preferred embodiment, a therapeutic gene, such as the native gene for dysferlin, is associated with treatment with a compound as described above, in particular bazedoxifene as a representative of third generation SERMs, namely compounds comprising a 2-phenylindole group. The administration of 2 treatments may be carried out simultaneously or at different times.

This treatment may also be combined with treatments which allow for an increase in muscle mass, such as the administration of decorin (WO2010/106295), lumican or fibromodulin (WO2013/072587).

The advantageous effects of bazedoxifene as a representative of the compounds as described above, in particular third generation SERMs, namely compounds comprising a 2-phenylindole group, result in an increase in the survival of muscle cells, in particular under osmotic shock conditions. These positive effects may be observed for the different skeletal muscles, both in an organism afflicted with a pathology associated with a muscle fragility as well as in a healthy individual. A priori, there are no secondary effects or immunological reactions to complain about.

Another possible application of compounds as described above, in particular bazedoxifene as a representative of third generation SERMs, namely compounds comprising a 2-phenylindole group, is the in vitro or ex vivo culture of myoblasts, myotubes or muscle fibers (or myofibers), for example as described by Tominaga et al. (iScience: 2021 Dec. 20; 25(1):103667). These cells or tissues may originate both from healthy organisms and from those afflicted with the aforementioned pathologies.

Unless indicated otherwise, the implementation of the present invention employs conventional techniques in molecular biology (including recombination techniques), in microbiology, in cell biology. in biochemistry and in immunology, which are known to the person skilled in the art. these techniques are explained in detail in the literature. in particular in “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting” (Babar, 2011); “Current Protocols in Immunology” (Coligan, 2002). Particularly useful techniques for particular embodiments will be discussed in the sections below.

The disclosures in each patent, patent application and publication cited in the present application are incorporated in their entirety herein by reference.

Without further description, it is considered that the person skilled in the art will be able to produce and use the compounds of the present invention and carry out the methods claimed using the description and the following illustrative examples.

### Exemplary Embodiments

The invention and advantages arising therefrom will become more apparent from the following exemplary embodiments, supported by the accompanying figures. They do not in any way limit the scope.

The invention is illustrated below with the aid of bazedoxifene, denoted Bazedoxifene, or bazedoxifene acetate, denoted Bazedoxifene acetate, tested on immortalized myoblasts.

### LEGENDS TO FIGURES

FIG. 1. Effect of Bazedoxifene on the quantity and localization of dysferlin L1341P

(A) Quantification of number of cells positive to dysferlin (●) and of viability (▪) after treatment of immortalized L1341P myoblasts with increasing concentrations of Bazedoxifene. The values were normalized by treatment with 0.1% DMSO and each point represents the mean±the standard deviation of four repetitions of a representative experiment (N=3).

(B) Analysis by immunofluorescence of the localization of dysferlin after treatment with 0.1% DMSO or the maximum effective dose (5 μM) of Bazedoxifene. The endoplasmic reticulum, the Golgi apparatus, the lysosomes and the autophagosomes were also identified with KDEL, GM130, Lamp1 and LC3B stains. The nuclei were stained with Hoechst (blue). Scale bar=10 μM.

FIG. 2. Measurement of protection of Bazedoxifene against osmotic shock

(A) Visualization and (B) quantification of mortality induced by osmotic shock on immortalized L1341P myoblasts after treatment with increasing concentrations of Bazedoxifene or 0.03% DMSO.

(C) Determination of the duration of osmotic shock (hours) necessary in order to induce 50% cell mortality.

Each point or bar represents the mean±the standard deviation of four repetitions of a representative experiment.

BZA=Bazedoxifene.

FIG. 3. Protective effect of Bazedoxifene independent of the mutation.

Measurement of the effect of 2 μbazedoxifene and/or bazedoxifene acetate on cell viability in response to osmotic shock in different immortalized myoblast lines carrying the mutations (A) L1341P (missense 1), (B) G299R (missense 2), (C) S1173X (ko) or (D) no mutation of dysferlin. Each point represents the mean±the standard deviation of four repetitions of a representative experiment.

### I) MATERIAL AND METHODS

### Cell Culture and Pharmacological Treatment

The immortalized myoblasts used in this study were obtained from patients afflicted with LGMDR2.

The principal cell line used (814) originated from a biopsy of the vastus lateralis muscle from a 57-year-old male patient carrying the following mutation: homozygous c.4022T>C: p.L1341P. The cells were isolated by the team of Dr Simone Spuler (Charité Medical University, Berlin) then immortalized by the team of Dr Vincent Mouly (Myological Institute, Paris). For the osmotic shock experiments, the immortalized myoblast lines 107 and AB320 used carried the following mutations:

heterozygous c.855+1delG, c.895G>A: mRNA decay, p.G299R and heterozygous c.342-1G>A, c.3516_3517delTT:/, p.Ser1173X.

The cells were cultured in a muscle cell proliferation medium: 1 volume of medium 199 (Invitrogen, 41150020) to 4 volumes of DMEM (Invitrogen, 61965-025), supplemented with 20% FCS, 25 μg/mL of fetuin (Life Technologies, 10344026), 5 ng/mL of hEGF (Life Technologies, PHG0311), 0.5 ng/ml of bFGF (Life Technologies, PHG0026), 0.2 μg/mL of dexamethasone (Sigma, D4902) and 5 μg/mL of insulin (Sigma, 91077). The cells were inoculated onto plates which had already been coated with 0.1% gelatin and kept under a controlled atmosphere at 37° C. and 5% CO₂.

Seventy-two hours after inoculation, the cells were treated with the compound bazedoxifene HCl or bazedoxifene acetate (Selleckchem) or dimethyl sulfoxide (DMSO, VWR) as a negative control. The cells were analyzed after 24 hours of treatment.

### Quantification of the Expression of Dysferlin, and Viability Test

After 24 hours of treatment, the cells were fixed with 4% paraformaldehyde (10 minutes, ambient temperature). Immunofluorescence was carried out in a phosphate buffered saline solution (PBS) supplemented with 0.1% of Triton for permeabilization (5 min, ambient temperature) then 1% bovine serum albumin (BSA: Sigma) for blocking (1 hour, ambient temperature). For the first hybridization step, the cells were incubated (overnight, 4° C.) with rabbit anti-dysferlin antibody (abcam, ab124684) and goat anti-desmine antibody (R&D, AF3844). The stains were then revealed during the second hybridization step by incubation (1 hour, ambient temperature) with secondary antibodies conjugated with a fluorochrome (Invitrogen, anti-rabbit green and anti-goat red) and the nuclei were stained with Hoechst (Invitrogen, 33342). The expression of the dysferlin was analyzed with the CellInsight CX7 imager (Cellomics Inc). The first channel was used to identify the nuclei, the second for the segmentation of the cells and the third for the identification and quantification of aggregates of dysferlin. The images were acquired with a 20× lens at high resolution and were analyzed with a colocalization bioapplication. By thresholding the intensity of the dysferlin, the number of cells positive to dysferlin staining (minimum one dysferlin aggregate in the cell) could be calculated in a manner such that it will be less than 10% with the negative control. The total number of cells was determined by counting the cells stained by Hoechst per well, which enabled the cell viability to be quantified by normalization with the negative control.

### Localization of Dysferlin

After 24 hours of treatment with Bazedoxifene (EC50=5 μM), the cells were fixed and stained (overnight, 4° C.) with the following primary antibodies: anti-dysferlin (Novocastra), Kdel (EnzoLife), GM130 (BD Biosciences), LAMP (Abcam) and LC3 A/B (ThermoScientific). After washing, the stains were then revealed with the aid of secondary antibodies coupled to fluorochromes (1 hour, ambient temperature) and analyzed with the aid of a confocal microscope (LSM 880, Carl Zeiss). The images were acquired with a 63× oil lens.

### Osmotic Shock

The cells were inoculated onto 96-well plates and treated with different concentrations of Bazedoxifene, as indicated, for 24 hours. The cells were then incubated for 3 hours with a red viability probe (1/3000, Incucyte® Nuclight Rapid Red Dye—Sartorius). Once the probe had been incorporated by the nuclei of living cells (Incucyte verification), an osmotic shock compound of 25% of PBS and 75% water was applied in the presence of a green mortality probe (1/3000, Incucyte® Caspase-3/7 Green Dye—Sartorius). The red and green staining was then observed every 90 minutes for 72 hours with an IncuCyte® S3 Live-Cell Analysis, thereby allowing the resistance of the cells to osmotic shock (ratio of green to red) to be measured after pharmacological treatment in comparison with a negative control (DMSO).

### II) RESULTS

### 1/Identification of Bazedoxifene as an Inhibitor of the Degradation of the Poorly Conformed Protein Dysferlin

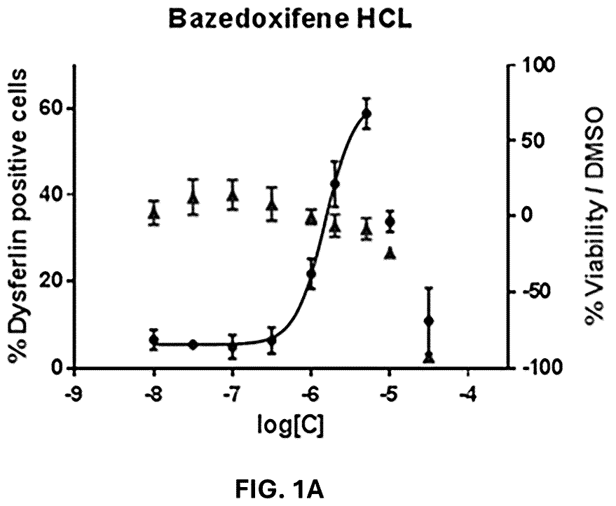

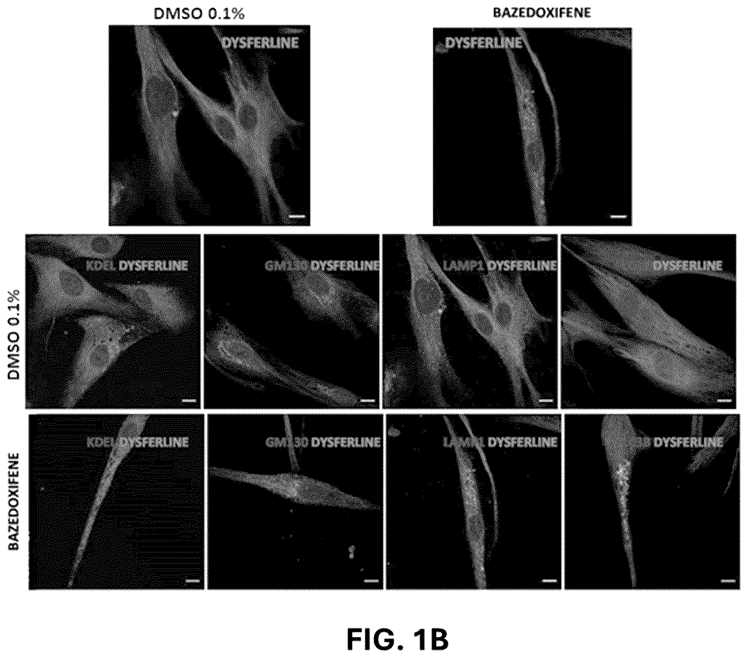

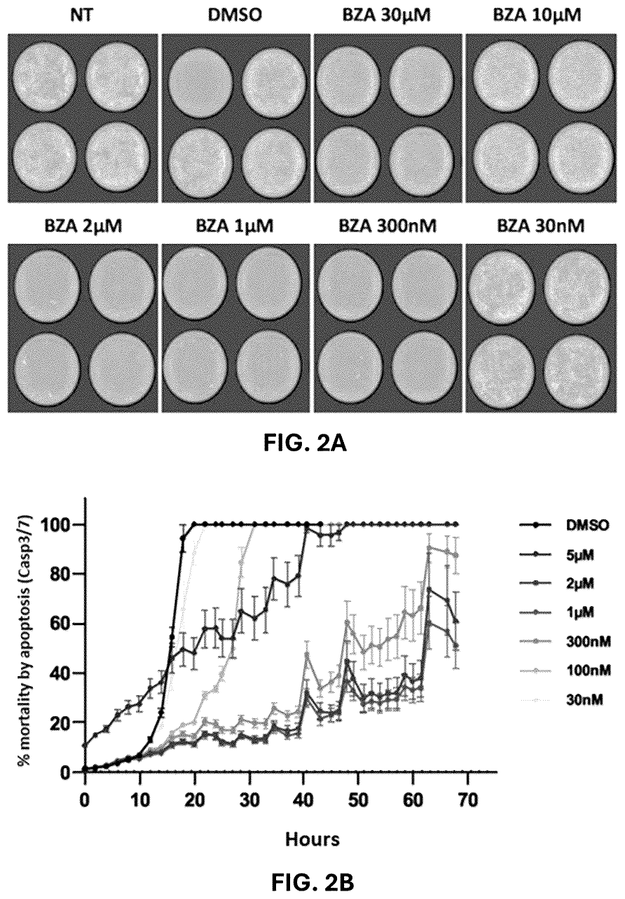

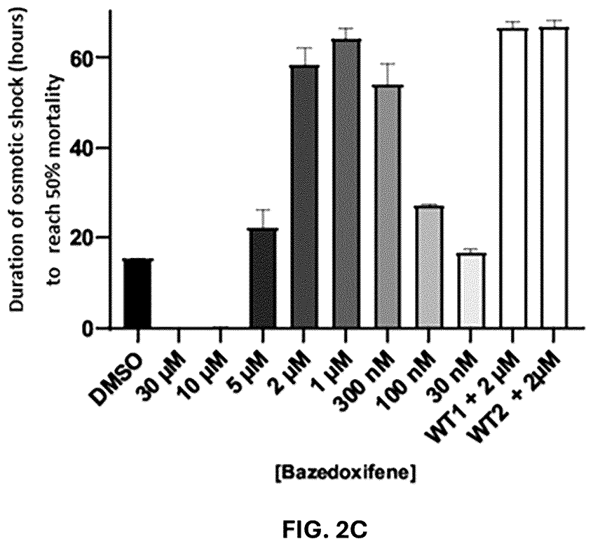

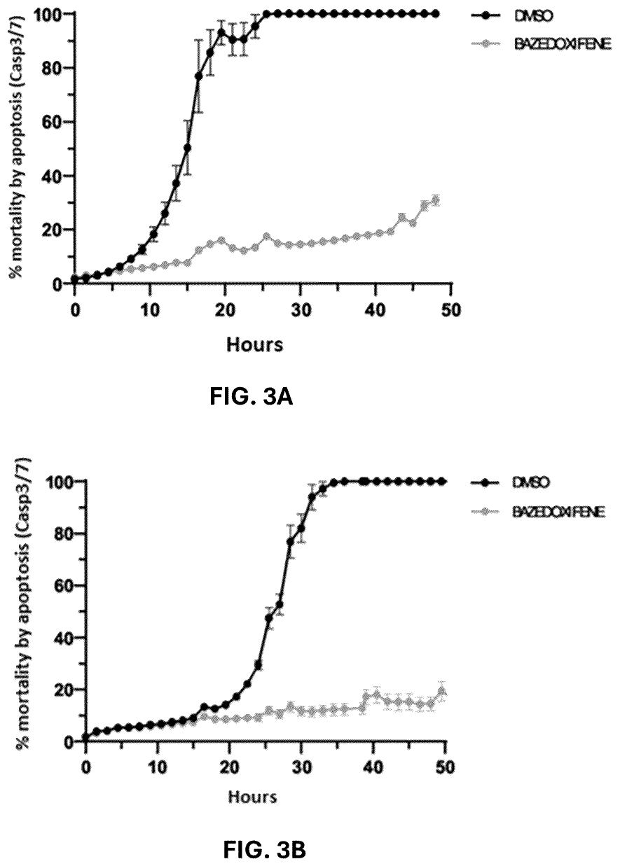

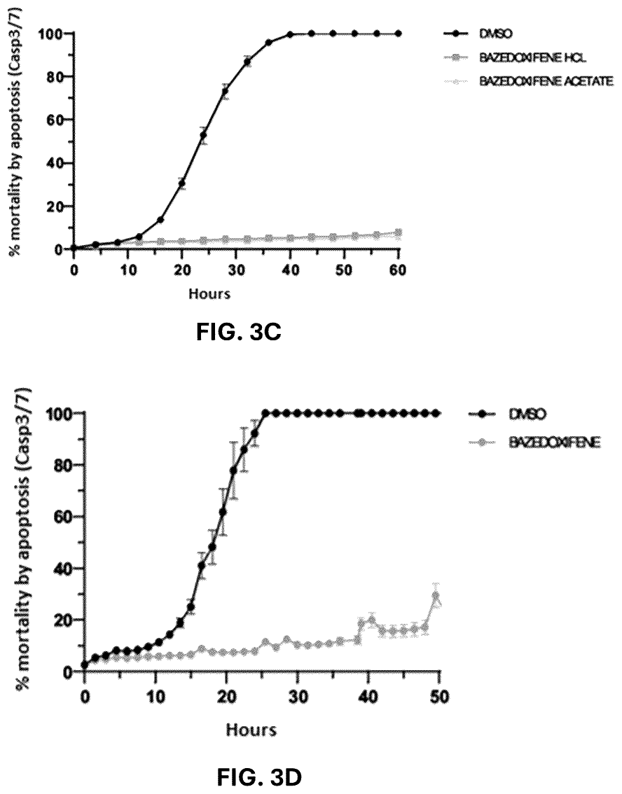

As demonstrated in FIG. 1A, the use of increasing doses of Bazedoxifene causes an increase in the number of mutated immortalized L1341P myoblasts expressing at least one aggregate of dysferlin, without revealing significant toxicity, except at very high doses. This effect was confirmed by the histological analysis shown in FIG. 1B: in the presence of the maximum effective dose of Bazedoxifene (5 μ), the quantity of dysferlin detected in the immortalized L1341P myoblasts was increased compared with the DMSO treatment control.

It has been demonstrated that the L1341P mutation causes the poor conformation of the dysferlin, its absence in the sarcolemma and its aggregation in the endoplasmic reticulum: thereby causing membrane fragility and in fine the death of muscle cells. The analysis of the localization of the L1341P dysferlin, by staining the different cell compartments in FIG. 1B, demonstrates the exit of the protein from the endoplasmic reticulum after treatment with Bazedoxifene.

On the basis of these experiments, the hypothesis can be formulated that Bazedoxifene prevents the degradation of the poorly conformed protein by the ERAD system, thereby increasing the quantity of functional dysferlin in the muscle cells and potentially its functionality at the membrane and cell survival.

### 2/Bazedoxifene Improves Cell Survival

In order to demonstrate a functionality of Bazedoxifene on the fragility of muscle cells, the survival of the treated immortalized L1341P myoblasts was evaluated after an osmotic shock which destabilized the cell membranes. FIGS. 2A and 2B demonstrate that at adapted doses of Bazedoxifene, the treatment of immortalized L1341P myoblasts results in an increase in cell viability under osmotic shock conditions. In addition, according to FIG. 2C, the survival of muscle cells is greatly improved after treatment with a dose of Bazedoxifene equivalent to EC50, which in particular causes an increase of more than 40% of the number of myoblasts positive to dysferlin (FIG. 1).

FIG. 3 confirms that the protective effect of Bazedoxifene (HCl or acetate) on cell survival under osmotic shock conditions, and thus on the viability of the muscle cells, results from a more general effect on the cells and not solely on the aggregated dysferlin. In fact, the immortalized myoblasts resist osmotic shock after treatment with Bazedoxifene in a similar manner irrespective of whether they carried the L1341P (missense 1), G299R (missense 2), S1173X (ko) mutations of dysferlin. Surprisingly, this same effect was observed in WT cells, i.e. immortalized non-mutated myoblasts in the gene for dysferlin.

The present application therefore reveals that Bazedoxifene is effective for a broad spectrum of conditions associated with a fragility of muscle cells, in particular linked to the integrity of their membranes.

1-15. (canceled)

16. A method of treating a pathology associated with a muscle fragility comprising administering an estrogen receptor modulator (SERM) comprising a 2-phenylindole group to a subject.

17. The method of claim 16, wherein the pathology is a neuromuscular disease or a cachexia.

18. The method of claim 16, wherein the pathology is a genetic disease.

19. The method of claim 16, wherein the pathology affects the skeletal muscles.

20. The method of claim 16, wherein the pathology is a muscular dystrophy.

21. The method of claim 20, wherein the muscular dystrophy is a dysferlinopathy.

22. The method of claim 16, wherein the SERM is bazedoxifene or bazedoxifene acetate.

23. The method of claim 16, wherein the SERM is in the form of a tablet.

24. The method of claim 23, wherein the tablet comprises 20 mg of bazedoxifene or bazedoxifene acetate.

25. The method of claim 16, wherein the SERM is administered orally.

26. The method of claim 16, further comprising a gene therapy.

27. The method of claim 16, further comprising a cell grafting therapy.

28. A method of improving muscle survival or muscle resistance to stress, the method comprising administering a selective estrogen receptor modulator (SERM) comprising a 2-phenylindole group to a subject.

29. The method of claim 28, wherein the improvement in muscle survival compensates for deterioration resulting from immobilization or ageing.

30. The method of claim 28, wherein the SERM is bazedoxifene or bazedoxifene acetate.

31. The method of claim 28, wherein the SERM is in the form of a tablet.

32. The method of claim 31, wherein the tablet comprises 20 mg of bazedoxifene or bazedoxifene acetate.

33. The method of claim 28, wherein the SERM is administered orally.

34. The method of claim 28, further comprising a gene therapy.

35. The method of claim 28, further comprising a cell grafting therapy.

36. A method comprising:
culturing myoblasts, myotubules, or muscle fibers; and
adding to the culture a selective estrogen receptor modulator (SERM) comprising a 2-phenylindole group.

37. The method of claim 36, wherein the SERM is bazedoxifene or bazedoxifene acetate.
